(19) United States
(12) Patent Application Publication
(10) Pub. No.: US 2022/0225638 A1
(43) Pub. Date: Jul. 21, 2022

(54) USE OF BENZOIC ACID AND ESSENTIAL OIL COMPOUNDS FOR IMPROVING GROWTH PERFORMANCE

(71) Applicant: DSM IP ASSETS B.V., Heerlen (NL)

(72) Inventors: Hong LIU, Kaiseraugst (CH); Jose-Otavio SORBARA, Kaiseraugst (CH); Jinlong WU, Kaiseraugst (CH); Jingcheng ZHANG, Kaiseraugst (CH)

(21) Appl. No.: 17/615,225

(22) PCT Filed: Jun. 2, 2020
(86) PCT No.: PCT/EP2020/065227

§ 371 Date: Nov. 30, 2021

(30) Foreign Application Priority Data
Jun. 7, 2019 (CN) PCT/CN2019/090447
Jul. 19, 2019 (CN) PCT/CN2019/096792

(51) Int. Cl. A23K 20/111; A23K 50/75

(52) U.S. Cl. CPC A23K 20/111; A23K 50/75

(57) ABSTRACT

This invention relates to a composition comprising benzoic acid, derivatives or metabolites thereof, optionally in combination with a mixture of at least one essential oil compounds selected from the group consisting of thymol, eugenol and piperine, for improving growth performance of an animal subjected to coccidiosis vaccine challenge, and use thereof.

The present invention relates to a novel use of a nutraceutical composition for animals, especially poultry, comprising as active ingredients benzoic acid, derivatives or metabolites thereof, optionally in combination with a mixture of at least one essential oil compound selected from the group consisting of thymol, eugenol and piperine.

The term “nutraceutical” as used herein denotes a usefulness in both the nutritional and pharmaceutical field of application. Thus, the nutraceutical compositions can find use as a complete animal feed (diet), as supplement to animal feed, and as pharmaceutical formulations for enteral or parenteral application which may be solid formulations, or liquid formulations.

It is known from EP-A-0 683 985 that animal feed compositions comprising benzoic acid or salts thereof can be used to minimize the emission of odoriferous ammonia from organic wastes, especially animal excrements and manure.

It is also known from EP2042041 that benzoic acid, derivatives or metabolites thereof in combination with a mixture of essential oils can be used to improve feed conversion ratio and/or daily weight gain and/or modulate gut flora in animals.

It has now been found surprisingly that in addition to the above function, benzoic acid, derivatives or metabolites thereof, optionally in combination with a mixture of at least one essential oil compound selected from the group consisting of thymol, eugenol and piperine, has the advantage of being able to improve growth performance of an animal subjected to coccidiosis vaccine challenge.

In the present invention, improvement of the growth performance can be represented by the increase of weight gain (WG) and/or increase of feed intake (FI) of the animals with supplementation of benzoic acid or derivatives or metabolites thereof, optionally in combination with a mixture of at least one essential oil compound selected from the group consisting of thymol, eugenol and piperine, compared to the animals without supplementation of the combination.

Accordingly, the present invention relates to a composition comprising benzoic acid, derivatives or metabolites thereof, optionally in combination with a mixture of at least one, preferably at least two, more preferably at least three essential oil compounds selected from the group consisting of thymol, eugenol and piperine, for improving growth performance of an animal subjected to coccidiosis vaccine challenge.

The present invention also relates to the novel use of the composition as defined above for improving growth performance of an animal subjected to coccidiosis vaccine challenge.

The invention further relates to a method for improving growth performance of an animal subjected to coccidiosis vaccine challenge. This method comprises administering to the animal an effective amount of the composition as defined above.

Benzoic acid is commercially available (for example as VevoVitall®, supplied by DSM Nutritional Products, Kaiseraugst, Switzerland) or can easily be prepared by a skilled person using processes and methods well-known in the prior art.

Specific examples of derivatives of benzoic acid which can give rise to benzoic acid in vivo include salts of benzoic acid such as the alkali metal-alkaline earth metal- and ammonium benzoates.

The essential oil compounds according to the invention are commercially available or can easily be prepared by a skilled person using processes and methods well-known in the prior art.

The essential oil compounds can be used in highly purified forms in mixtures or in the form of natural available plant extracts or extract-mixtures.

The term “extract” as used herein includes compositions obtained by solvent extraction (which are also known as “extracted oils”), steam distillation (which are also known as “essential oils”) or other methods known to the skilled person. Suitable extraction solvents include alcohols such as ethanol.

By the expression “natural” is in this context understood a substance which consists of compounds occurring in nature and obtained from natural products or through synthesis. The natural substance may preferably contain at least one, preferably at least two of the compounds as defined above as main ingredient and additionally other essential oil compounds as for example capsaicin, tannin or carvacrol.

One example of the mixture of essential oils is CRINA® Poultry which is commercially available from DSM Nutritional Products (Switzerland).

Preferably, the composition according to the present invention comprises benzoic acid, derivatives or metabolites thereof.

More preferably, the composition according to the present invention comprises benzoic acid, derivatives or metabolites thereof, in combination with a mixture of thymol, eugenol and piperine. One example of the composition comprising benzoic acid in combination with a mixture of thymol, eugenol and piperine is CRINA° Poultry Plus which is commercially available from DSM Nutritional Products (Switzerland).

The compositions in the present invention may be in form of an animal feed or an additive or a premix therefore.

The amount of benzoic acid or of a derivative thereof administered to the animal may be in the range from 0.001% to 5% based on the total weight of each animal feed fed to the animal. This amount may, however, be higher if the function of benzoic acid or a derivative thereof is also to control the pH of the animal excreta fed on such a diet in order to suppress the emission of ammonia from the excreta. Such higher amounts are suitably limited to a maximum of about 10% based on the total animal feed composition.

The amount of benzoic acid or a derivative thereof in the final animal feed is preferably in the range of from 50 mg/kg to 1000 mg/kg feed, more preferably in the range of from 100 mg/kg to 500 mg/kg feed, and most preferably in the range of from 200 mg/kg to 250 mg/kg feed.

In a preferred embodiment of a poultry feeding concept, benzoic acid or a derivative of benzoic acid being used in an amount sufficient to provide a daily dosage of from 5 mg per kg body weight to about 80 mg per kg body weight, preferably from 10 mg per kg body weight to about 40 mg per kg body weight, of the subject to which it is to be administered.

The three active compounds thymol, eugenol and piperine may be administered in amounts of from 0.1 mg to 20 mg per kg feed (ppm), preferably in the range of from 0.3 mg to 10 mg per kg feed.

Examples of particularly preferred dosages of the three compounds in a final feed are independently from each other in the following ranges:
thymol between 1 ppm and 20 ppm, preferably between 1 ppm and 10 ppm;
eugenol between 1 ppm and 5 ppm, for example 2 ppm;
piperine up to 1 ppm, preferably between 0.3 ppm and 0.5 ppm.

The composition according to the present invention includes optionally other chemical compounds, for example at least one compound found in plants, and selected from the following group, as, per kg of feed,
up to about 1 mg of propylidene, butylidene, phtalides, gingerol, and/or lavender oil;
up to about 2 mg of decalactones, undecalactones, dodecalactones, ionones, irone, resorcinol, eucalyptol, menthol, peppermint oil, and/or alpha-pinene;
up to about 3 mg of limonene, guajacol, anethol, linalool, and/or methyl dihydrojasmonate;
up to about 4 mg of carvacrol, propionic, acetic or butyric acid, rosemary oil, clove oil, geraniol, terpineol, and/or citronellol;
up to about 5 mg of amyl, benzyl salicylate, cinnamaldehyde, and/or vanilline, a plant polyphenol (tannin); and/or
and up to about 5 mg of a powder of turmeric or of an extract of curcuma.

All these additional compounds may be used in combination with an emulsifying surfactant which may be selected advantageously from those of a rather hydrophilic nature, for example among polyglycerol esters of fatty acids such as esterified ricinoleic acid or propylene glycol esters of fatty acids, saccharo-esters or saccharo-glycerides, polyethylene glycol, lecithins etc.

In a preferred embodiment of a poultry feeding concept, the composition according to the present invention includes benzoic acid and a mixture of thymol, eugenol and piperine; wherein these three compounds being used in amounts sufficient to provide a daily dosage of 0.1 mg to about 1 mg thymol and eugenol and of 0.02 mg to about 0.06 mg piperine per kg body weight of the subject to which it is to be administered.

Apart from the above ingredients, the composition according to the present invention contains at least one fat soluble vitamin, and/or at least one water soluble vitamin, and/or at least one trace mineral, and/or at least one macro mineral.

Examples of fat-soluble vitamins include but are not limited to vitamin A, vitamin D₃, vitamin E, and vitamin K, e.g. vitamin K₃.

Examples of water-soluble vitamins include but are not limited to vitamin B₁₂, biotin and choline, vitamin B₁, vitamin B₂, vitamin B₆, niacin, folic acid and panthothenate, e.g. Ca-D-panthothenate.

Examples of trace minerals include but are not limited to manganese, zinc, iron, copper, iodine, selenium, and cobalt.

Examples of macro minerals include but are not limited to calcium, phosphorus and sodium.

The term “animal” in the present invention includes all animals including human. Examples of animals are non-ruminants, and ruminants. Ruminant animals include, for example, animals such as sheep, goat, and cattle, e.g. cow such as beef cattle and dairy cows. Preferably, the animal is a non-ruminant animal. Examples of non-ruminant animals include but are not limited to horses, rabbits; pig or swine (including but not limited to, piglets, growing pigs, and sows) and poultry. Especially preferably, the animal is poultry.

In the present invention, the term “poultry” refers to domesticated birds kept by humans for their eggs, their meat or their feathers, including but not limited to turkeys, ducks and chickens (including but not limited to broiler chicks, layers), goose, pigeon.

The present invention is further illustrated with reference to the following Example, which shows the effects of benzoic acid on weight gain, feed intake and feed conversion ratio in broiler chickens.

### EXAMPLE

### Material and Methods

Experimental Design and Diets

A total of 800 day-old male broiler chickens (Arbor Acres Plus) were sorted by body weight and randomly assigned to 4 dietary treatments each with 8 replicates and 25 birds per pen. The treatments including a non-challenge group (PC), a challenge group (NC), NC supplemented with benzoic acid (1000 mg/kg feed), and NC supplemented with CRINA® Poultry Plus (300 mg/kg feed, DSM Nutritional Products, Switzerland). The diets were formulated based on wheat, corn and soybean meal. All diets were in pelleted form and formulated according to the recommendation of China NY/T33-2004. The basal diets were prepared in mash form as indicated in Table 1, then the basal diet was subdivided into 4 experimental diets, appropriate amounts of tested feed additives were mixed with a small quantity of basal diet as a premix which was then added to the feed to get the final concentration, according to the treatment. After mixing, the feed was pelleted at 75-80° C.

TABLE 1
Ingredient and nutrient compositions of basal diets

Basal diets

| | Starter | Grower |
|---|---|---|
| Ingredients (%) | (0 to 21 d) | (21 to 35 d) |
| Wheat | 40.00 | 50.00 |
| Corn | 22.77 | 17.33 |
| Soybean meal | 29.82 | 24.75 |
| Soybean oil | 3.07 | 4.20 |
| L-Lysine HCl | 0.15 | 0.12 |
| DL-Met | 0.21 | 0.10 |
| L-threonine | 0.03 | 0.02 |
| Sodium bicarbonate | 0.24 | 0.22 |
| Limestone | 1.26 | 1.17 |
| DCP | 1.70 | 1.37 |
| NaCl | 0.25 | 0.22 |
| Premix¹ | 0.50 | 0.50 |
| Total | 100.00 | 100.00 |
| Calculated nutrients | | |
| & energy content | | |
| ME, kcal/kg | 2950 | 3050 |
| CP, % | 21.50 | 20.00 |
| Ca, % | 1.00 | 0.90 |
| tP, % | 0.69 | 0.63 |
| nPP, % | 0.45 | 0.40 |
| Lys, % | 1.15 | 1.01 |
| SAA, % | 0.91 | 0.76 |
| Thr, % | 0.81 | 0.73 |
| Trp, % | 0.28 | 0.25 |
| Na, % | 0.20 | 0.18 |
| Analyzed nutrient content, % | | |
| CP | 22.56 | 20.13 |
| Ca | 1.06 | 0.93 |
| tP | 0.73 | 0.61 |

¹Vitamin-mineral premix provided (per kg of diet) = Vitamin A 8250 IU, Vitamin D₃ 2187.5 IU, 25-OH-D₃ 69 μg, Vitamin E 41.25 IU, Vitamin K₃ 5 mg, Vitamin B₁ 2.5 mg, Vitamin B₂ 8.75 mg, Vitamin B₆ 5 mg, Vitamin B₁₂ 0.025 mg, Biotin 0.25 mg, Folic acid 2.5 mg, Niacinamide 50 mg, D-pantothenic acid 15 mg, Fe 120 mg, Cu 10 mg, Mn 110 mg, Zn 90 mg, I 0.5 mg, Se 0.25 mg, Choline 400 mg.

Birds Management and Sampling

The birds were reared in floor pens on fresh wood shaving bedding in an environmentally controlled room and received 23L:1D of lighting program during the first week and 20L:4D afterward until the end of the study. The temperature of the room was maintained at 32±1° C. on the day of arrival and adapted according to the age specific requirements of the chicks. Birds were offered starter diets during 0-21 d and grower diet during 21-35 d. Feed and water were provided ad libitum. At 4 days of age, the birds in challenge groups (NC) were received a coccidial vaccine (Coccivac B®, Merck Animal Health), which contained live oocysts of E. tenella, E. mivati, E. maxima and E. acervuline, with 2-fold dose in a 0.5 ml sterile water by gavage. Because of the small volume, non-challenged birds were not given a placebo gavage. Pen weight and feed intake were measured at day-old, 21 and 35 days of age, dead animals were monitored twice daily and the weights of dead birds were recorded for the calculation of FCR.

Statistical Analysis

The experiment was a completely randomized design with 4 treatments. The data were analyzed using the General Linear Model procedure of SPSS (version 22.0). Each pen served as an experimental unit. Contrast procedures were used to compare PC to NC, NC to the treatment with Benzoic acid or CRINA® Poultry Plus at P<0.05.

Results

The growth performance of broiler receiving different dietary treatments are presented in Table 2.

TABLE 2
Growth performance of broilers receiving different
dietary treatments from days 0 to 35 of age

| Treatment | Description | FI, g/bird | WG, g/bird | Mortality, % |
|---|---|---|---|---|
| T1 | PC | 3072 | 1913 | 5.5 |
| T2 | NC | 3021 | 1901 | 5.0 |
| T3 | Benzoic acid | 3071 | 1930 | 6.0 |
| T4 | CRINA ® Poultry | 3162 | 1959 | 4.5 |
| | Plus | | | |

In the overall period, CRINA® Poultry Plus significantly increased feed intake and weight gain while decreasing the mortality of the birds.

The clinic symptoms of a coccidiosis infection are related with intestinal mucosal damage, poor feed utilization and growth depression. In the current trial, the success of coccidial vaccine challenge was evidenced by the impaired feed intake and weight. The supplementation of CRINA° Poultry Plus could compensate this negative effect and the values reached to that of the PC.

Conclusions

Coccidial vaccine challenge results in growth depression while supplementation of benzoic acid or mixture of benzoic acid and essential oils improves growth performance of broiler chickens subjected to coccidiosis challenge.

1. A composition comprising benzoic acid, derivatives or metabolites thereof, optionally in combination with a mixture of at least one essential oil compounds selected from the group consisting of thymol, eugenol and piperine, for improving growth performance of an animal subjected to coccidiosis vaccine challenge.

2. The composition of claim 1, wherein the animal is poultry including but not limited to turkeys, ducks and chickens (including but not limited to broiler chicks, layers), goose, pigeon.

3. The composition of claim 1, which is in form of an animal feed or an additive or a premix therefore.

4. Use of the composition of any one of claims 1 for improving growth performance of an animal subjected to coccidiosis vaccine challenge.

5. Use of claim 4, wherein the animal is poultry including but not limited to turkeys, ducks and chickens (including but not limited to broiler chicks, layers), goose, pigeon.

6. A method for improving growth performance of an animal subjected to coccidiosis vaccine challenge comprising administering to the animal an effective amount of the composition of claim 1.

7. The method of claim 6, wherein the animal is poultry including but not limited to turkeys, ducks and chickens (including but not limited to broiler chicks, layers), goose, pigeon.

8. The method of claim 6, wherein benzoic acid, derivatives or metabolites thereof is administered in an amount of from 50 mg/kg to 1000 mg/kg feed, preferably in the range of 100 mg/kg to 500 mg/kg, most preferably in the range of 200 mg/kg to 250 mg/kg feed.

9. The method of claim 6, wherein benzoic acid, derivatives or metabolites thereof is administered in an amount sufficient to provide a daily dosage of 5 mg per kg body weight to about 80 mg per kg body weight, preferably 10 mg per kg body weight to about 40 mg per kg body weight, of the animal.

10. The method of claim 6, wherein the essential oil compounds are administered in amounts of from 0.1 mg to 20 mg per kg feed (ppm), preferably in the range of from 0.3 mg to 10 mg per kg feed.
